(19) United States
(12) Patent Application Publication
(10) Pub. No.: US 2011/0178349 A1
(43) Pub. Date: Jul. 21, 2011

(54) METHOD OF TREATING A HYDROCARBON STREAM COMPRISING CYCLOPENTADIENE AND ONE OR MORE DIOLEFINS

(76) Inventors: Robert Kenneth ANZICK, Kemah, TX (US); Robert Lawrence BLACKBOURN, Houston, TX (US); SivaKumar NAYARAJAN, Katy, TX (US)

(21) Appl. No.: 12/969,299

(22) Filed: Dec. 15, 2010

Related U.S. Application Data
(60) Provisional application No. 61/296,664, filed on Jan. 20, 2010.

(51) Int. Cl. C07C 2/02

(52) U.S. Cl. 585/318; 585/360

(57) ABSTRACT

A method of treating a hydrocarbon stream comprising cyclopentadiene (CPD) and one or more diolefins, the method comprising: providing a hydrocarbon stream comprising an initial CPD concentration, an initial target diolefin concentration, and an initial secondary diolefin concentration; subjecting the hydrocarbon stream to preseparation conditions effective to separate (a) a crude target diolefin feedstock having a decreased concentration of CPD and an increased target diolefin concentration, and (b) a CPD dimerization feedstock having a decreased target diolefin concentration and an increased CPD concentration that is 1.5 or more times the initial CPD concentration; subjecting the CPD dimerization feedstock to CPD dimerization conditions effective to produce a raw DCPD stream comprising 20 wt. % or more DCPD; and, separating a high purity DCPD product stream comprising 90 wt. % or more DCPD from the raw DCPD stream.

This application claims the benefit of Provisional Application No. 61/296,664 filed Jan. 20, 2010, which is incorporated herein by reference.

### FIELD OF THE INVENTION

The present application provides a method of treating a hydrocarbon stream comprising cyclopentadiene (CPD) and one or more diolefins. More particularly, the present application provides a method of treating such a hydrocarbon stream to recover one or more of high purity dicyclopentadiene (DCPD), a raw target diolefins stream, and/or a resin grade secondary diolefins stream.

### BACKGROUND OF THE INVENTION

Diolefins, such as isoprene (IP) and piperylenes (PIPS), typically are recovered from hydrocarbon streams that also comprise CPD. Advantageously, CPD tends to react with itself to form dicyclopentadiene (DCPD), which may be recovered as a valuable product. CPD in a hydrocarbon stream also tends to undergo undesirable reactions at moderate to elevated temperatures. For example, CPD tends to react with isoprene to form a CPD-IP co-dimer and, to a lesser extent, with piperylenes to form a CPD-PIPS co-dimer. Further reactions producing oligomers, for example, trimers and tetramers also may occur. Examples include the reaction between CPD and DCPD, CPD and the CPD-IP co-dimer, and CPD and the CPD-PIPS co-dimer. CPD also tends to react with other reactive compounds that may be found in the hydrocarbon stream.

These undesirable reactions produce undesirable products, which can cause downstream processing problems. For example, these undesirable products can cause fouling of equipment, contamination of processing materials such as extraction solvents, increased operating costs, and/or contamination of the resulting products. Because of this, CPD generally is regarded as a contaminant in hydrocarbon streams serving as feedstocks for the recovery of diolefins. In particular, CPD is regarded as a contaminant in the feedstocks processed for the recovery of high purity isoprene and/or piperylenes.

Various methods have been used to avoid the foregoing problems. Some remove CPD by subjecting the hydrocarbon stream to dimerization conditions effective to dimerize CPD in the stream to produce DCPD. For example, a C5 hydrocarbon stream containing CPD typically is heated to dimerize the CPD, thus enabling recovery of a “low purity DCPD stream” comprising 75 wt % or less DCPD. The value of the process is reduced because the low purity DCPD stream has relatively less value on the market than would a “high purity DCPD stream,” or a DCPD stream comprising 90 wt % or more DCPD.

It is difficult to obtain a “high purity DCPD stream” by directly dimerizing a low purity hydrocarbon stream comprising CPD. It is even more difficult to obtain a high purity DCPD stream by directly dimerizing a C5 hydrocarbon stream. In order to obtain a high purity DCPD stream, the low purity DCPD stream produced by dimerizing a C5 hydrocarbon stream generally is upgraded.

Unfortunately, upgrading of a low purity DCPD stream can be complex and expensive. Upgrading usually involves heating the low purity DCPD stream to a sufficiently high temperature to crack or monomerize the DCPD back to CPD, selectively recovering the CPD from the cracking process, and dimerizing the recovered CPD once again to produce a higher purity DCPD stream. This method suffers from high fouling of equipment due to the high temperatures involved in the cracking process and the fouling characteristics of DCPD at high temperatures. Alternately, upgrading may involve a series of relatively complex vacuum distillation and purification steps. This method suffers from the high number of separation devices required and the potential ingress of oxygen into the process and subsequent fouling associated with oxygen reacting with DCPD and CPD.

Even when upgrading is performed, the process lacks efficiency because at least some of the CPD in the starting hydrocarbon stream typically is not converted and recovered as high purity DCPD. Some of the CPD in the hydrocarbon stream may react during the dimerization process to produce oligomers that are heavier than DCPD, such as trimers and/or tetramers. The oligomers generally do not revert back to CPD in the upgrading process. Some of the CPD in the hydrocarbon stream also may not be converted to DCPD during the dimerization process. Unconverted CPD often is lost within the downstream processes, such as high purity isoprene recovery processes, through “destructive” removal before or within the isoprene recovery plant. The result is a net loss of CPD and lower conversion to DCPD.

Some have attempted to produce higher purity DCPD solely by dimerizing a hydrocarbon feedstock or C5 hydrocarbon stream at lower temperatures. It is true that, at lower dimerization temperatures, undesirable competing reactions between components of the hydrocarbon feedstock or C5 hydrocarbon stream occur at slower rates. However, satisfactory conversions of CPD to DCPD are not achieved at lower temperatures unless the residence time is increased. Longer residence times are achieved through larger equipment volumes and through increase(s) in the initial cost of process equipment and/or additional equipment installation.

Some have attempted to produce higher purity DCPD by recovering DCPD at sub-atmospheric pressures. However, the use of sub-atmospheric pressures can lead to oxygen ingress into the process and subsequent fouling.

Others have attempted to upgrade low purity DCPD via monomerizing or cracking the DCPD back to CPD; separating the CPD from the remaining DCPD and heavier components at, near, or below atmospheric conditions; dimerizing the recovered CPD; and, finally separating the remaining CPD and DCPD. Unfortunately, such processes tend to be complex and costly.

Methods are needed for treating hydrocarbon streams comprising CPD and one or more diolefins to produce a high purity DCPD stream without requiring vacuum distillation and the associated possible ingress of oxygen into the process, without requiring excessively long dimerization residence times, and without requiring complex thermal cracking of low purity DCPD streams and/or subsequent dimerization of recovered CPD.

### SUMMARY OF THE INVENTION

The present application provides a method of treating a hydrocarbon stream comprising CPD and one or more diolefins to preseparate a CPD rich stream and a diolefins rich stream.

In one embodiment, the method further comprises processing the CPD rich stream and/or the diolefins rich stream to recover one or more of high purity DCPD, a raw target diolefins stream, and/or a resin grade secondary diolefins stream.

In one embodiment, the present application provides a method of treating a C5 hydrocarbon stream comprising CPD and isoprene to preseparate a CPD rich stream and an isoprene rich stream.

In one embodiment, the method further comprises processing the CPD rich stream and/or the isoprene rich stream to recover one or more of high purity DCPD, a raw isoprene stream, and/or a resin grade piperylenes stream.

In one embodiment, the present application provides a method of treating a hydrocarbon stream comprising cyclopentadiene (CPD) and one or more diolefins, the method comprising: providing a hydrocarbon stream comprising an initial CPD concentration, an initial target diolefin concentration, and an initial secondary diolefin concentration; subjecting the hydrocarbon stream to preseparation conditions effective to separate (a) a crude target diolefin feedstock having a decreased concentration of CPD and an increased target diolefin concentration, and (b) a CPD dimerization feedstock having a decreased target diolefin concentration and an increased CPD concentration that is 1.5 or more times the initial CPD concentration; subjecting the CPD dimerization feedstock to CPD dimerization conditions effective to produce a raw DCPD stream comprising 20 wt. % or more DCPD; and, separating a high purity DCPD product stream comprising 90 wt. % or more DCPD from the raw DCPD stream.

### BRIEF DESCRIPTION OF THE DRAWINGS

One or more embodiments of the invention are described in detail and by way of example only with reference to the accompanying drawing wherein:

FIG. 1 is a schematic process flow diagram of one embodiment of the method.

### DETAILED DESCRIPTION OF THE INVENTION

The present application provides a method of treating a hydrocarbon stream comprising CPD and one or more diolefins to preseparate a CPD rich stream and a diolefins rich stream.

In one embodiment, the method further comprises processing the CPD rich stream and/or the diolefins rich stream to recover one or more of high purity DCPD, a raw target diolefins stream, and/or a resin grade secondary diolefins stream.

In one embodiment, the present application provides a method of treating a C5 hydrocarbon stream comprising CPD and isoprene to preseparate a CPD rich stream and an isoprene rich stream. In one embodiment, the method further comprises processing the CPD rich stream and/or the isoprene rich stream to recover one or more of high purity DCPD, a raw isoprene stream, and/or a resin grade piperylenes stream.

In one embodiment, the method avoids the need for complex upgrading of low purity DCPD streams. In one embodiment, the method avoids the need for substantial additional equipment. In one embodiment, the method avoids the potential for a run-away reaction possible with DCPD-cracking methods and processes. In one embodiment, the method reduces the formation of unwanted byproducts or contaminants, which can foul processing equipment.

In one embodiment, the method produces high purity DCPD without requiring vacuum distillation. In one embodiment, the method produces high purity DCPD without requiring an increase in dimerization residence time. In one embodiment, the method produces high purity DCPD without inducing oxygen ingress into the dimerization process. In one embodiment, the method produces high purity DCPD and without complex thermal cracking of low purity DCPD streams and subsequent dimerization of the recovered CPD. In one embodiment, the CPD dimerization temperature may be lower than the temperatures required to dimerize an unseparated hydrocarbon stream. The CPD dimerization feedstock also comprises a reduced diolefin content compared to an unseparated hydrocarbon stream. These factors result in decreased formation of impurities during CPD dimerization and increased selectivity to DCPD. In one embodiment, the method produces high purity DCPD without the need for substantial additional equipment. In one embodiment, the method produces high purity DCPD without the potential for a run-away reaction possible with DCPD-cracking methods and processes. In one embodiment, the method produces high purity DCPD without the need for complex upgrading of low purity DCPD streams.

In one embodiment, a raw target diolefins stream is produced. In this embodiment, the method has the advantage that net target diolefins in the crude target diolefins feedstock are increased. In one embodiment, the method results in greater recovery of target diolefins than would be achieved processing an unseparated hydrocarbon stream. In this embodiment, the raw target diolefins stream produced generally has sufficient quality for use as a feedstock to high purity diolefins recovery processes. In one embodiment, the method reduces the formation of unwanted byproducts and/or contaminants.

In one embodiment, a raw isoprene stream is produced. In this embodiment, the method has the advantage that net isoprene in the crude isoprene feedstock is increased. In one embodiment, the method results in greater recovery of isoprene than would be achieved by treating an unseparated C5 hydrocarbon stream. In this embodiment, the raw isoprene stream produced generally has sufficient quality for use as a feedstock to high purity isoprene recovery processes. In one embodiment, the method reduces the formation of unwanted byproducts and/or contaminants.

In one embodiment, the method processes the diolefins rich stream to produce a resins grade secondary olefins product. In one embodiment, the method produces a resins grade piperylenes product.

Without limiting the application to a particular theory of operation, it is believed that the method achieves the foregoing by increasing the concentration of a target substrate in the feed to a process for recovering the target substrate and/or a reaction product of the target substrate. In one embodiment, the method increases the concentration of CPD in the feedstock to CPD dimerization en route to the recovery of DCPD. In one embodiment, the method increases the concentration of target diolefins in the crude target diolefin feedstock en route to recovery of the target diolefins. In one embodiment, the method increases the concentration of secondary diolefins in the crude secondary diolefins feedstock en route to recovery of the secondary diolefin. Increasing the concentration of the respective substrate in the respective feedstocks is believed to increase conversion of the respective substrate to the desired product. This is because the rate of reaction of two or more chemical species generally is a function of: (a) the concentration of reactants; (b) the operating temperature; and to a much lesser extent (c) the operating pressure.

The method will be better understood from the following description:

### The Separations, Generally

The separators or fractionators referred to in the following description may be substantially any structure in which the feedstock may be separated into the described streams. Suitable structures may include, for example, distillation columns. Suitable distillation columns may contain internals for assisting in the separation. Suitable internals include, for example, one or more trays, packing, or combinations thereof. A distillation column generally has a number of theoretical stages that may vary. Persons of ordinary skill in the art will be able to determine the optimum number of stages for the particular separation using standard procedures. Generally, as the number of stages increases, operating flexibility increases, variability in feed composition may be accommodated, and generally higher purity products may be recovered.

In one embodiment, the separators or fractionators comprise a reboiler to provide heat for vapor generation and a condenser to condense vapor for generation of liquid reflux and distillate. A variety of heat sources for reboiling may be used, including steam or heat integration with other parts of the process. Heat is removed from the condenser by any suitable cooling medium including cooling water, air, refrigerant, or heat integration with other parts of the process.

The separations or fractionations may be batch or continuous. If the separation is continuous, the separation may be carried out in a single or in multiple separators. Where multiple separators are used, any number of separators can be employed.

The separations or fractionations generally occur under conditions comprising a bottoms temperature, an overhead temperature, and a top operating pressure, which vary relative to one another. The conditions may comprise substantially any operating temperatures, depending on the operating pressure. The conditions also may comprise substantially any operating pressure, depending upon the operating temperature.

### The Reactors, Generally

The reactors may have a variety of constructions and any number of reactors may be employed either in series or in parallel. For example, the reactors may be horizontal or vertical in construction. In one embodiment, one or more reactors are horizontal in construction. In one embodiment, the CPD dimerization reactor and the crude piperylenes feedstock dimerization reactor are horizontal in construction with a series of vertical baffles. The reactors may be operated in a variety of modes such as completely liquid filled or with a vapor space above the liquid. In one embodiment, the CPD dimerization reactor and the crude piperylenes feedstock dimerization reactor are horizontal in construction with a series of vertical baffles and with a vapor space above the liquid.

### The Hydrocarbon Stream

The hydrocarbon stream processed according to the present method may originate from any suitable source. One of the main sources of a hydrocarbon stream comprising CPD is a pyrolysis gasoline stream produced during the manufacture of ethylene. The following detailed description refers to the processing of a pyrolysis gasoline stream. However, the description is for purposes of illustration only and should not be construed as limiting the claims to processing of such a hydrocarbon stream. For example, other suitable streams may originate from the upgrading of low purity DCPD streams or other hydrocarbon streams that contain an initial concentration of CPD and diolefins in which the advantage is gained through preseparation of the CPD and removal of other possible reactants such as isoprene, piperylenes and/or other reactive hydrocarbons.

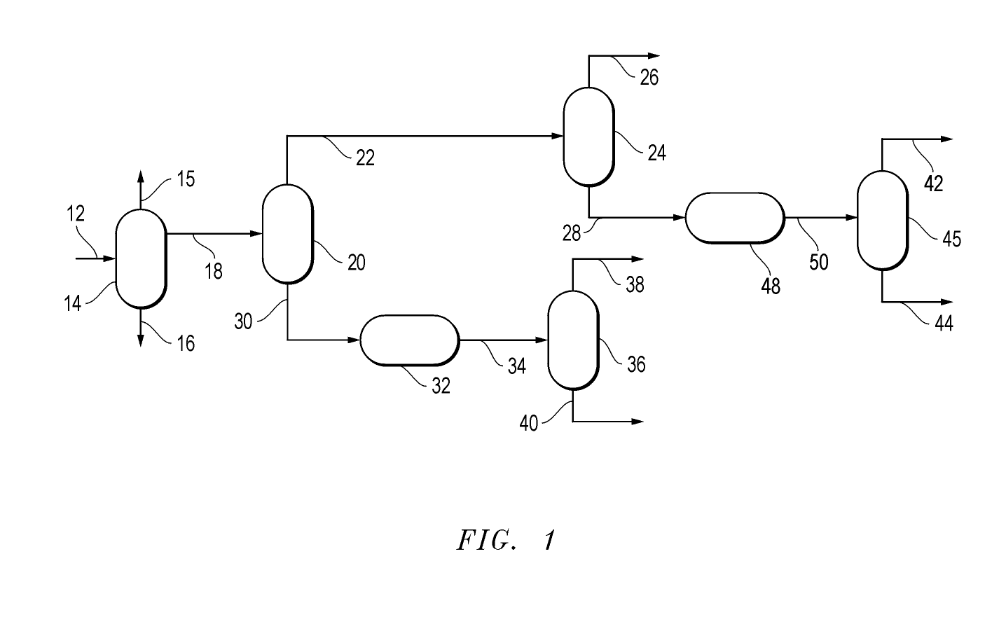

The method will be described further with reference to FIG. 1. FIG. 1 is a schematic diagram of an embodiment of the method, which is for illustrative purposes only and should not be considered as limiting the claims to a particular embodiment. In particular, reference to isoprene and piperylenes is for illustrative purposes only, and should not be considered as limiting the claims to a particular embodiment.

Referring to FIG. 1, a pyrolysis gasoline stream 12 comprises C5+ hydrocarbons including, for example, paraffins, olefins, and diolefins. The pyrolysis gasoline stream 12 typically is separated into C4 and lighter stream 15, a crude C5 feedstock 18 and a C6+ hydrocarbon stream 16. The C6+ hydrocarbon stream 16 may be further processed to recover aromatics such as benzene and toluene. The crude C5 feedstock 18 may be processed to recover C5-specific compounds, for example, isoprene, piperylenes, and dicyclopentadiene through dimerization of CPD. The crude C5 feedstock 18 also may be processed with the C6+ hydrocarbon stream 16, and ultimately hydrotreated or directly hydrotreated and added to the refinery gasoline pool.

The predominate products produced when the crude C5 feedstock 18 is processed to recover C5-specific compounds include, for example, isoprene (2-methyl-1,3-butadiene, or “IP”), piperylenes (cis- and trans-1,3-pentadiene, or “PIPS”), and dicyclopentadiene or “DCPD”. Other compounds include, for example, isoamylene, which is typically a mixture of 2-methyl-butene-1 (“2 MB1”) and 2-methyl-butene-2 (“2MB2”).

Some DCPD exists in and can be directly recovered from a pyrolysis gasoline stream 12. The main source of produced DCPD is from dimerization of CPD, which is found in the recovered crude C5 feedstock 18. At moderate temperatures and pressures, CPD molecules will react with other CPD molecules via a Diels-Alder reaction mechanism to form DCPD. Various purity-grades of DCPD can be produced, depending on the quality and origin of the pyrolysis gasoline stream, the impurities in the pyrolysis gasoline stream, the operating conditions employed in the CPD dimerization process, and the processing equipment and schemes used during the DCPD manufacturing process.

### Preseparation

In FIG. 1, the crude C5 feedstock 18 is fed to a preseparator 20. In the preseparator 20, the crude C5 feedstock 18 is subjected to preseparator conditions effective to produce a crude isoprene feedstock 22. The preseparator conditions also are effective to simultaneously or separately produce an improved CPD dimerization feedstock 30. In FIG. 1, the improved CPD dimerization feedstock 30 is recovered at or near the bottom of the preseparator 20.

Because much of the isoprene, paraffins, and some of the olefins and diolefins are removed from the crude C5 feedstock 18 in the preseparator 20, the CPD concentration in the CPD dimerization feedstock 30 is greater than the CPD concentration in the crude C5 feedstock 18. In one embodiment, the CPD concentration in the CPD dimerization feedstock 30 is at least 1.5 or more times the CPD concentration in the C5 hydrocarbon stream, or the crude C5 feedstock 18 in FIG. 1. In one embodiment, the CPD concentration in the CPD dimerization feedstock 30 is 2 or more times the CPD concentration in the C5 hydrocarbon stream, or the crude C5 feedstock 18 in FIG. 1.

In one embodiment, the isoprene concentration in the CPD dimerization feedstock 30 is 2.9 or more times less than the isoprene concentration in the C5 hydrocarbon stream, or the crude C5 feedstock 18 in FIG. 1. In one embodiment, the isoprene concentration in the CPD dimerization feedstock 30 is 5 or more times less than the isoprene concentration in the C5 hydrocarbon stream, or in the crude C5 feedstock 18 in FIG. 1. In one embodiment, the isoprene concentration in the CPD dimerization feedstock 30 is 10 or more times less than the isoprene concentration in the C5 hydrocarbon stream, or in the crude C5 feedstock 18 in FIG. 1. In one embodiment, the isoprene concentration in the CPD dimerization feedstock 30 is 15 or more times less than the isoprene concentration in the C5 hydrocarbon stream, or the crude C5 feedstock 18 in FIG. 1.

In one embodiment, the ratio of CPD to isoprene in the CPD dimerization feedstock 30 is 7 or more. In one embodiment, the ratio of CPD to isoprene in the CPD dimerization feedstock 30 is 10 or more. In one embodiment, the ratio of CPD to isoprene is 20 or more. In one embodiment, the ratio of CPD to isoprene is 30 or more.

In one embodiment, the preseparation conditions comprise a mass reflux ratio of about 4.9 or more, based on the ratio of reflux to crude C5 feedstock 18. In one embodiment, the preseparation conditions comprise a mass reflux ratio of about 4.3 or more on the same basis. In one embodiment, the preseparation conditions comprise a mass reflux ratio of about 4.1 or less on the same basis. In one embodiment, the preseparation conditions comprise a mass reflux ratio of about 3.6 or more on the same basis.

In one embodiment, the preseparator conditions comprise a preseparator pressure of about 34.5 kPa (5 psig) or more. In one embodiment, the preseparator conditions comprise a preseparator pressure of about 110 kPa (16 psig) or less. In one embodiment, the preseparator conditions comprise a preseparator pressure of about 82 kPa (12 psig) or less.

In one embodiment, the preseparator conditions comprise a preseparator temperature of about 40° C. (105° F.) or more. In one embodiment, the preseparator conditions comprise a preseparator temperature of about 51° C. (123.8° F.) or less.

In an advantageous embodiment, the preseparation conditions comprise a preseparator top operating pressure of about 34.5 kPa (5 psig), a preseparator bottoms temperature of about 50.6° C. (123° F.), and a preseparator overhead temperature of about 40.6° C. (105° F.).

In the embodiment described in FIG. 1, a crude isoprene feedstock 22 is recovered at or near the top of the preseparator 20. In one embodiment, additional hydrocarbon streams or C5 hydrocarbon streams containing relatively substantial amounts of CPD may be added to the crude C5 feedstock 18 to increase the CPD concentration of the stream before subjecting the stream to preseparator conditions.

### Dimerization of CPD

In FIG. 1, a CPD dimerization feedstock 30 obtained from at or near the bottom of the preseparator 20 is fed to a CPD dimerization reactor 32. In the CPD dimerization reactor 32, the CPD dimerization feedstock 30 is subjected to CPD feedstock dimerization conditions effective to produce a raw DCPD stream 34. The concentration of CPD in the CPD dimerization feedstock 30 is relatively higher than that found in the crude C5 feedstock 18 in FIG. 1. As a result, in the conversion of CPD to DCPD relatively high selectivity to DCPD may be achieved with lower CPD dimerization temperatures than would be used to directly dimerize CPD in the crude C5 feedstock 18 or other C5 hydrocarbon streams. Undesirable reactions between CPD and isoprene that otherwise would compete with the dimerization of CPD are reduced due to the lower temperatures and the relatively low concentration of isoprene. Undesirable reactions between CPD and piperylenes or CPD and any other reactive hydrocarbons that otherwise would compete with the dimerization of CPD are reduced due to the relatively low temperature of the CPD dimerization conditions.

The CPD dimerization conditions, or the conditions at which the CPD in the CPD dimerization feedstock 30 is dimerized, are generally used in the CPD dimerization reactor 32 and in the crude piperylenes dimerization reactor 48 shown in FIG. 1.

The CPD dimerization conditions may comprise substantially any effective CPD dimerization temperature, depending on the CPD dimerization pressure. The CPD dimerization conditions also may comprise substantially any effective CPD dimerization pressure, depending upon the CPD dimerization temperature. In one embodiment, the CPD dimerization conditions comprise a CPD dimerization operating temperature that is sufficiently low to minimize decomposition of DCPD into CPD. In one embodiment, the CPD dimerization conditions comprise a CPD dimerization operating temperature that is sufficiently low to minimize reaction between CPD, isoprene, piperylenes, and/or other reactive hydrocarbons and/or to minimize additional reaction of CPD with DCPD. In one embodiment, the CPD dimerization conditions comprise a CPD dimerization temperature of about 40.5° C. (105° F.) or more. In one embodiment, the CPD dimerization conditions comprise a CPD dimerization temperature of about 71° C. (160° F.) or less.

In one embodiment, the CPD dimerization conditions comprise a CPD dimerization pressure of about 345 kPa (50 psig) or less. In one embodiment, the CPD dimerization conditions comprise a CPD dimerization pressure of about 207 kPa (30 psig) or more. In one embodiment, the CPD dimerization conditions comprise atmospheric pressure, or about 103 kPa (15 psig) or more.

In one embodiment, the CPD dimerization conditions comprise a CPD dimerization pressure of about 207 kPa (30 psig), a CPD dimerization inlet temperature of about 40.5° C. (105° F.), and a CPD dimerization outlet temperature of about 71° C. (160° F.).

The CPD dimerization conditions also comprise a CPD dimerization residence time. In one embodiment, the CPD dimerization residence time is about 5 hours or more. In one embodiment, the CPD dimerization residence time is about 7.5 hours or more. In one embodiment, the CPD dimerization residence time is about 10.5 hours or less. In one embodiment, the CPD dimerization residence time is from about 5 to about 10.5 hours.

### High Purity DCPD Recovery

In FIG. 1, the raw DCPD stream 34 is fed to a raw DCPD separator 36. In the raw DCPD separator 36, the raw DCPD stream 34 is subjected to DCPD separation conditions effective to recover a high purity DCPD product 40. In FIG. 1, the high purity DCPD product 40 is recovered at or near the bottom of the raw DCPD separator 36. In FIG. 1, the DCPD separation conditions also are effective to produce a supplemental crude piperylenes (“PIPS”) feedstock 38. In FIG. 1, the supplemental crude PIPS feedstock 38 is recovered at or near the top of the raw DCPD separator 36.

In one embodiment, the supplemental crude PIPS feedstock 38 is combined with the crude isoprene feedstock 22 and subjected to further treatment as described below. In one embodiment, the complimentary crude PIPS feedstock 38 is combined with the crude PIPS feedstock 28 and subjected to further treatment as described below. In one embodiment, the supplemental crude PIPS feedstock 38 is separately subjected to conditions similar to the post fractionation conditions to which the crude PIPS feedstock 28 is subjected.

Referring back to the raw DCPD separator 36, the DCPD separation conditions are effective to produce a concentration of DCPD in the high purity DCPD product (40 shown in FIG. 1) which is higher than would be produced by dimerizing a C5 hydrocarbon stream (such as the crude C5 feedstock 18) which has not been subjected to preseparation. For example, a DCPD product produced by dimerizing a C5 hydrocarbon stream which has not been subjected to preseparation (an “unseparated C5 hydrocarbon stream”) typically would comprise from about 75 to about 80 wt. % DCPD. In contrast, the high purity DCPD product (such as, for example, 40 in FIG. 1) produced by dimerizing a preseparated C5 hydrocarbon stream, or a C5 hydrocarbon stream which has been subjected to preseparation according to the present method, generally comprises 90 wt. % or more DCPD. In one embodiment, the high purity DCPD product comprises 94.5 wt. % or more DCPD.

In one embodiment, the DCPD separation conditions comprise a DCPD separation mass reflux ratio of about 4.9 or more, based on the ratio of reflux to the raw DCPD stream 34 in FIG. 1.

The DCPD separation conditions may comprise substantially any operating temperatures, depending on the operating pressure. The DCPD separation conditions also may comprise substantially any operating pressure, depending upon the operating temperatures. In one embodiment, the DCPD separation conditions comprise operating temperatures and pressures that are sufficiently low to minimize decomposition of DCPD into CPD. In one embodiment, the DCPD separation conditions comprise operating temperatures and pressures that are sufficiently high to minimize the ingress of oxygen as can occur in sub-atmospheric or vacuum conditions.

In one embodiment, the DCPD separation conditions comprise a DCPD separation pressure of about 27.5 kPa (4 psig) or more. In one embodiment, the DCPD separation conditions comprise a DCPD separation pressure of about 55 kPa (8 psig) or more. In one embodiment, the DCPD separation conditions comprise a DCPD separation pressure of about 34 kPa (5 psig) and a DCPD separator overhead temperature of from about 50° C. (122° F.).

Vacuum distillation may be used in conjunction with the methods described herein. However, it is not necessary to use vacuum distillation in order to achieve the results described herein.

For example, the high purity DCPD stream produced by preseparation is efficiently produced by dimerization at: (a) relatively low dimerization temperatures; (b) relatively low dimerization pressures; (c) relatively small dimerization residence times; and, (d) relatively small dimerization reactor volumes.

High purity DCPD streams generally are produced by dimerization of CPD. High purity DCPD streams are desirable because they generally may be used to produce higher value products.

For example, DCPD streams comprising from about 60 to 75 wt. % DCPD typically are used to manufacture relatively low value hydrocarbon (petroleum) resins. DCPD streams comprising from about 75 to 85 wt % DCPD typically are used to produce somewhat higher value unsaturated polyester resins (UPR). DCPD streams comprising from about 92 to 95 wt. % DCPD may be used to ultimately produce relatively high value ethylene propylene diene monomer (EPDM) rubbers. DCPD streams comprising greater than about 95 wt. % DCPD typically are used to produce the relatively high value Reaction Injection Molding (RIM) products.

Recovery of Raw Isoprene Stream and/or

### Resin Grade PIPS Product

In one embodiment, the crude isoprene feedstock 22 recovered from the preseparator 20 is fed directly to a high purity isoprene recovery process as a raw isoprene feedstock. In one embodiment, the crude isoprene feedstock 22 recovered from the preseparator 20 is fed directly to a high purity isoprene recovery process comprising extractive distillation using acetonitrile solvent. Persons of ordinary skill in the art will be familiar with suitable processing and operating conditions for such high purity isoprene recovery processes.

In the embodiment illustrated in FIG. 1, the crude isoprene feedstock 22 recovered from the preseparator 20 is fed to a prefractionator 24. In the prefractionator 24, the crude isoprene feedstock 22 is subjected to prefractionator conditions effective to produce a raw isoprene stream 26 and a crude PIPS feedstock 28.

In one embodiment, referring to FIG. 1, the raw isoprene stream 26 is recovered at or near the top of the prefractionator 24. In FIG. 1, the crude PIPS feedstock 28 is recovered at or near the bottom of the prefractionator 24. The supplemental crude PIPS feedstock 38 produced in the raw DCPD separator 36 may be fed to the prefractionator 24 or to the crude PIPS dimerization reactor 48 for further processing as described herein.

In one embodiment, the prefractionator conditions comprise a mass reflux ratio of about 8.0 or more, based on the ratio of reflux to feed to the crude isoprene feedstock 22. In one embodiment, the prefractionator conditions comprise a mass reflux ratio of about 9.0 or more on the same basis. In one embodiment, the prefractionator conditions comprise a mass reflux ratio of about 12.0 or less on the same basis. In one embodiment, the prefractionator conditions comprise a mass reflux ratio of about 11.0 or less on the same basis. In one embodiment, the prefractionator conditions comprise a mass reflux ratio of about 10.0 on the same basis.

In one embodiment, the prefractionator conditions comprise a prefractionator pressure of about 103 kPa (15 psig) or more. In one embodiment, the prefractionator conditions comprise a prefractionator pressure of about 138 kPa (20 psig) or more. In one embodiment, the prefractionator conditions comprise a prefractionator pressure of about 345 kPa (50 psig) or less. In one embodiment, the prefractionator conditions comprise a prefractionator pressure of about 172 kPa (25 psig) or less. In one embodiment, the prefractionator conditions comprise a prefractionator pressure of about 35 kPa (5 psig).

In one embodiment, the prefractionator conditions comprise a prefractionator top operating pressure of about 35 kPa (5 psig), a prefractionator bottoms temperature of about 50.6° C. (123° F.), and a prefractionator overhead temperature of about 40.6° C. (105° F.).

—Production of Resin Grade PIPS Product

In one embodiment, the crude PIPS feedstock 28 is subjected to crude PIPS treatment conditions effective to produce a resin grade PIPS product. In one embodiment, the crude PIPS treatment conditions also produce a low purity DCPD product. In one embodiment, the low purity DCPD product is fed to the preseparator 20 for further processing depending on the desired DCPD product recovery and purity.

—Crude PIPS/CPD Dimerization

In one embodiment, illustrated in FIG. 1, the crude PIPS feedstock 28 is fed to a crude PIPS/CPD dimerization reactor 48. In one embodiment, the crude PIPS feedstock 28 is subjected to crude PIPS/CPD dimerization conditions effective to produce a raw PIPS stream 50 comprising dimerized CPD (DCPD) and PIPS.

The crude PIPS/CPD dimerization conditions may comprise substantially any effective CPD dimerization temperature, depending on the crude PIPS/CPD dimerization pressure. The crude PIPS/CPD dimerization conditions also may comprise substantially any effective CPD dimerization pressure, depending upon the crude PIPS/CPD dimerization temperature. In one embodiment, the crude PIPS/CPD dimerization conditions comprise a crude PIPS/CPD dimerization operating temperature that is sufficiently low to minimize decomposition of DCPD to CPD. In one embodiment, the crude PIPS/CPD dimerization conditions comprise a crude PIPS/CPD dimerization operating temperature that is sufficiently low to minimize reaction between piperylenes and/or other reactive hydrocarbons. In one embodiment, the crude PIPS/CPD dimerization operating temperature is about 68° C. (155° F.) or more. In one embodiment, the crude PIPS/CPD dimerization operating temperature is about 82° C. (180° F.) or less. In one embodiment, the crude PIPS/CPD dimerization operating temperature is about 70° C. (158° F.) or more. In one embodiment, the crude PIPS/CPD dimerization operating temperature is about 80° C. (176° F.) or less.

In one embodiment, the crude PIPS/CPD dimerization conditions comprise a crude PIPS/CPD dimerization pressure of about 345 kPa (50 psig) or less. In one embodiment, the crude PIPS/CPD dimerization conditions comprise a crude PIPS/CPD dimerization pressure of about 207 kPa (30 psig) or more. In one embodiment, the crude PIPS/CPD dimerization conditions comprise atmospheric pressure, or about 103 kPa (15 psig).

In one embodiment, the crude PIPS/CPD dimerization conditions comprise a crude PIPS/CPD dimerization pressure of about 207 kPa (30 psig), a crude PIPS/CPD dimerization inlet temperature of about 40.5° C. (105° F.), and a crude PIPS/CPD dimerization outlet temperature of about 71° C. (160° F.).

The crude PIPS/CPD dimerization conditions also comprise a crude PIPS/CPD dimerization residence time. In one embodiment, the crude PIPS/CPD dimerization residence time is about 5 hours or more. In one embodiment, the crude PIPS/CPD dimerization residence time is about 7.5 hours or more. In one embodiment, the crude PIPS/CPD dimerization residence time is about 10.5 hours or less. In one embodiment, the crude PIPS/CPD dimerization residence time is from about 5 to about 10.5 hours.

—Post-Fractionation of Raw PIPS Stream

In one embodiment, illustrated in FIG. 1, the raw PIPS stream 50 recovered from the crude PIPS/CPD dimerization reactor 48 is fed to post-fractionator 45. In one embodiment, the raw PIPS stream 50 is subjected to post-fractionation conditions effective to produce a resins grade PIPS product 42 and a low purity DCPD product 44. A “resins grade piperlyenes product” is defined to mean a PIPS product having a combined cis-1,3-pentadiene and trans-1,3-pentadiene concentration typically greater than 55 wt %.

In one embodiment, the postfractionation conditions comprise a postfractionation pressure of about 55 kPa (8 psig) or more. In one embodiment, the postfractionation conditions comprise a postfractionation pressure of about 103 kPa (15 psig) or more. In one embodiment, the postfractionation conditions comprise a postfractionation pressure of about 138 kPa (20 psig) or more. In one embodiment, the postfractionation conditions comprise a postfractionation pressure of about 345 kPa (50 psig) or less. In one embodiment, the postfractionation conditions comprise a postfractionation pressure of about 172 kPa (25 psig) or less. The mass reflux ratio of the post-fractionation may be adjusted to achieve a desired purity.

In one embodiment, the post-fractionation conditions comprise a post-fractionation top operating pressure of about 55 kPa (8 psig), a post-fractionation bottoms operating temperature of about 68° C. (154° F.), and a post-fractionation overhead operating temperature of about 55° C. (131° F.).

In one embodiment, illustrated in FIG. 1, the resin grade PIPS product 42 is recovered at or near the top of the post-fractionator 45. In one embodiment, the low purity DCPD product 44 is recovered at or near the bottom of the post-fractionator 45.

The foregoing detailed description relates to the separation of a C5 hydrocarbon stream to recover a crude isoprene feedstock and a CPD dimerization feedstock. The detailed description is illustrative only, and the present method also may be used, for example, to treat other streams comprising CPD and diolefins. Given the foregoing description, persons of ordinary skill in the art will be able to modify known processes for treating such streams to achieve similar results.

The application will be better understood with reference to the following Examples, which are illustrative only and should not be construed as limiting the claims. The simulations in the following Examples were performed using an accepted industry simulation tool, such as PROII Simulation Software from SimSci-Esscor.

### Example 1

A simulation was performed using a simulated C5 hydrocarbon stream 18 having the starting composition and feed rate given in the following Table. The simulation subjected a simulated C5 hydrocarbon stream 18 to simulated preseparation conditions. The simulated preseparation conditions comprised a simulated preseparation pressure of 34.5 kPa (5 psig), a preseparator bottoms temperature of about 50.6° C. (123° F.), a preseparator overhead temperature of about 40.6° C. (105° F.), and a mass reflux ratio of about 3.6 with respect to the C5 hydrocarbon stream 18. The simulated preseparation conditions produced a simulated CPD dimerization feedstock 30 having the composition given in the following Table.

The simulation subjected the simulated CPD dimerization feedstock 30 to simulated dimerization conditions. The simulated dimerization conditions comprised a simulated dimerization feed rate given in the following Table, a simulated dimerization pressure of about 338 kPa (50 psig), a simulated dimerization inlet feed temperature of about 40° C. (105° F.), a simulated outlet temperature of about 71° C. (160° F.), and a simulated dimerization residence time of about 7.8 hours. The simulated dimerization conditions produced a raw DCPD stream 34 having the composition given in the following Table:

| | | | Simulated | | Simulated | |
|---|---|---|---|---|---|---|
| | Simulated C5 | | CPD Dimerization | | Raw DCPD | |
| | Hydrocarbon Stream | | Feedstock | | Stream | |
| | (18) | | (30) | | (34) | |
| | Simulated | Simulated | Simulated | Simulated | Simulated | Simulated |
| | Feed Rate | Composition | Feed Rate | Composition | Feed Rate | Composition |
| Component | (lb/hr) | (wt %) | (lb/hr) | (wt %) | (lb/hr) | (wt %) |
| Cyclopentadiene | 5657.7 | 16.0 | 5091.9 | 31.3 | 923.3 | 5.7 |
| Isoprene | 6755.9 | 19.2 | 650.8 | 4.0 | 623.1 | 3.8 |
| Trans-1,3-Pentadiene | 3427.9 | 9.7 | 3276.4 | 20.1 | 3247.6 | 20.0 |
| Cis-1,3-Pentadiene | 2229.8 | 6.3 | 2190.3 | 13.5 | 2190.3 | 13.5 |
| DCPD | 0.0 | 0.0 | 0.0 | 0.0 | 4113.8 | 25.3 |
| Co-Dimers | 0.0 | 0.0 | 0.0 | 0.0 | 111.4 | 0.7 |
| Other C5's | 17192.7 | 48.8 | 5061.7 | 31.1 | 5061.6 | 31.0 |
| Total | 35264.0 | 100.0 | 16271.1 | 100.0 | 16271.1 | 100.0 |

As seen from the Table, the concentration of CPD in the simulated CPD dimerization feedstock 30 increased by a factor of 1.9 relative to the concentration of CPD in the simulated C5 hydrocarbon stream 18. The concentration of isoprene in the simulated CPD dimerization feedstock 30 decreased by a factor of 4.8 relative to the concentration of isoprene in the C5 hydrocarbon stream 18. The ratio of CPD to isoprene in the simulated CPD dimerization feedstock 30 was about 7.8 compared to a ratio of 0.8 CPD to isoprene in the C5 hydrocarbon stream 18.

The concentration of CPD in the simulated raw DCPD stream 34 decreased by a factor of 2.8 relative to the concentration of CPD in the simulated C5 hydrocarbon stream 18. The concentration of isoprene in the simulated raw DCPD stream 34 decreased by a factor of 5.1 relative to the concentration of isoprene in the simulated C5 hydrocarbon stream 18.

### Example 2

In this example, a simulation was performed in which the simulated raw DCPD stream (34) having the starting composition and feed rate shown in the preceding Table was subjected to simulated DCPD separation conditions. The simulated DCPD separation conditions comprised a simulated DCPD separation pressure of about 55 kPa (8 psig), a simulated DCPD separation top operating temperature of about 54° C. (130° F.), a simulated DCPD separation bottom operating temperature of about 170° C. (338° F.), and a simulated DCPD separation mass reflux ratio of about 11.5 with respect to the raw DCPD stream 34. The simulated DCPD separation conditions produced a simulated high purity DCPD product 40 with a simulated DCPD concentration of at least 94.5 wt. %.

### Example 3

In this example, a simulation was performed in which a simulated crude isoprene feedstock 22 having the composition and feed rate given in the following Table was combined with a simulated crude PIPS feedstock 38 produced in Example 2 to produce a simulated raw isoprene stream 26 having the composition and feed rate given in the following Table.

The simulation subjected the combined simulated crude isoprene feedstock 22 to simulated prefractionation conditions. The simulated prefractionation conditions comprised a simulated prefractionation pressure of about 55 kPa (8 psig), a simulated prefractionation top operating temperature of about 45° C. (113° F.), a simulated prefractionation bottom operating temperature of about 68° C. (155° F.), and a simulated prefractionation mass reflux ratio of about 10.1 with respect to the combined crude isoprene feedstock 22 and crude PIPS feedstock 38. The simulated prefractionation conditions produced a simulated raw isoprene stream 26 and a simulated crude PIPS stream 28 having the following composition:

| | Simulated Crude | | Simulated Crude | | Simulated Raw | | Simulated Crude | |
|---|---|---|---|---|---|---|---|---|
| | Isoprene | | PIPS | | Isoprene | | PIPS | |
| | Feedstock | | Feedstock | | Stream | | Stream | |
| | (22) | | (38) | | (26) | | (28) | |
| | Simulated | Simulated | Simulated | Simulated | Simulated | Simulated | Simulated | Simulated |
| | Feed Rate | Composition | Feed Rate | Composition | Feed Rate | Composition | Feed Rate | Composition |
| Component | (lb/hr) | (wt %) | (lb/hr) | (wt %) | (lb/hr) | (wt %) | (lb/hr) | (wt %) |
| Cyclopentadiene | 565.8 | 3.0 | 921.7 | 7.7 | 26.1 | 0.1 | 604.4 | 5.4 |
| Isoprene | 6105.1 | 32.1 | 622.8 | 5.2 | 6673.1 | 33.9 | 20.1 | 0.2 |
| Trans-1,3-Pentadiene | 151.5 | 0.8 | 3239.4 | 27.2 | 55.3 | 0.3 | 3298.1 | 29.3 |
| Cis-1,3-Pentadiene | 39.4 | 0.2 | 2179.1 | 18.3 | 13.1 | 0.1 | 2205.5 | 19.6 |
| DCPD | 0.0 | 0.0 | 0.0 | 0.0 | 0.0 | 0.0 | 787.7 | 7.0 |
| Co-Dimers | 0.0 | 0.0 | 0.0 | 0.0 | 0.0 | 0.0 | 142.1 | 1.3 |
| Other C5's | 12131.1 | 63.9 | 4955.5 | 41.6 | 12895.6 | 65.6 | 4190.4 | 37.2 |
| Total | 18992.9 | 100.0 | 11918.5 | 100.0 | 19663.2 | 100.0 | 11248.3 | 100.0 |

As seen from the Table, the content of CPD in the simulated crude isoprene stream 22 decreased by a factor of 22 on a weight basis compared to the content of CPD in the simulated raw isoprene stream 26. The content of isoprene in the simulated raw isoprene stream 26 increased slightly compared to the crude isoprene stream 22.

The contents of other components in the simulated raw isoprene stream 26 were sufficiently low that the simulated raw isoprene stream 26 was suitable for processing directly in a high purity isoprene recovery process comprising an extractive distillation system utilizing acetonitrile solvent.

### Example 4

In this example, a simulated crude PIPS stream 28 having the starting composition and feed rate given in the following Table was subjected to simulated dimerization conditions. The simulated dimerization conditions comprised a simulated dimerization pressure of about 530 kPa (77 psig), a simulated dimerization inlet feed temperature of about 68° C. (155° F.), a simulated outlet temperature of about 82° C. (180° F.), and a simulated dimerization residence time of about 9.0 hours. The simulated dimerization conditions produced a raw PIPS stream 50 having a composition and rate in the following Table. The raw PIPS stream 50 was further subjected to simulated postfractionation conditions. The simulated postfractionation conditions comprised a simulated postfractionation pressure of about 55 kPa (8 psig), a simulated postfractionation top operating temperature of about 55° C. (131° F.), a simulated postfractionation bottom operating temperature of about 68° C. (154° F.), and a simulated postfractionation mass reflux ratio of about 27.0 with respect to the raw PIPS stream 50. The simulated postfractionation conditions produced a simulated resin grade PIPS product 42 having the following simulated composition and rate:

| | Simulated Crude | | Simulated Raw | | Simulated Resin | |
|---|---|---|---|---|---|---|
| | PIPS | | PIPS | | Grade PIPS | |
| | Stream | | Stream | | Product | |
| | (28) | | (50) | | (42) | |
| | Simulated | Simulated | Simulated | Simulated | Simulated | Simulated |
| | Feed Rate | Composition | Feed Rate | Composition | Feed Rate | Composition |
| Component | (lb/hr) | (wt %) | (lb/hr) | (wt %) | (lb/hr) | (wt %) |
| Cyclopentadiene | 604.4 | 5.4 | 222.3 | 2.0 | 222.1 | 2.3 |
| Isoprene | 20.1 | 0.2 | 19.6 | 0.2 | 19.6 | 0.2 |
| Trans-1,3-Pentadiene | 3298.0 | 29.3 | 3281.2 | 29.2 | 3274.6 | 34.1 |
| Cis-1,3-Pentadiene | 2205.5 | 19.6 | 2205.5 | 19.6 | 2189.6 | 22.8 |
| DCPD | 787.7 | 7.0 | 1152.2 | 10.2 | 0.0 | 0.0 |
| Co-Dimers | 142.1 | 1.3 | 176.3 | 1.6 | 0.0 | 0.0 |
| Other C5's | 4190.1 | 37.2 | 4190.8 | 37.2 | 3883.9 | 40.6 |
| Total | 11247.9 | 100.0 | 11247.9 | 100.0 | 9589.8 | 100.0 |

Persons of ordinary skill in the art will recognize that many modifications may be made to the embodiments described herein. The embodiments described herein are meant to be illustrative only and should not be taken as limiting the invention, which will be defined in the claims.

What is claimed is:

1. A method of treating a hydrocarbon stream comprising cyclopentadiene (CPD) and one or more diolefins, the method comprising:
providing a hydrocarbon stream comprising an initial CPD concentration, an initial target diolefin concentration, and an initial secondary diolefin concentration;
subjecting the hydrocarbon stream to preseparation conditions effective to separate (a) a crude target diolefin feedstock having a decreased concentration of CPD and an increased target diolefin concentration, and (b) a CPD dimerization feedstock having a decreased target diolefin concentration and an increased CPD concentration that is 1.5 or more times the initial CPD concentration;
subjecting the CPD dimerization feedstock to CPD dimerization conditions effective to produce a raw DCPD stream comprising 20 wt. % or more DCPD; and,
separating a high purity DCPD product stream comprising 90 wt. % or more DCPD from the raw DCPD stream.

2. The method of claim 1 wherein:
the preseparation conditions comprise a preseparation temperature of from about 40° C. to about 50° C.; and,
a preseparation pressure of from about 34.5 kPa to about 110 kPa.

3. The method of claim 1 wherein the decreased target diolefin concentration is 2.9 or more times less than the initial target diolefin concentration.

4. The method of claim 1 wherein the ratio of the concentration of CPD to the concentration of target diolefin in the CPD dimerization feedstock is about 7 or more.

5. The method of claim 1 wherein the CPD dimerization conditions comprise:
a CPD dimerization temperature of from about 40.5° C. to about 71° C.;
a CPD dimerization pressure of from about 103 kPa to about 345 kPa; and,
a CPD dimerization residence time of from about 5 hours to about 10.5 hours.

6. The method of claim 5 wherein the DCPD separation conditions comprise:
a DCPD separation temperature of from about 50° C. to about 170° C.; and
a DCPD separation pressure of from about 28 kPa to about 55 kPa.

7. The method of claim 1 further comprising subjecting the crude target diolefin feedstock to prefractionation conditions effective to separate (a) a raw target diolefins stream comprising a further increased concentration of the target diolefin and a further decreased concentration of CPD and secondary diolefin, and (b) a crude secondary diolefin feedstock comprising a decreased concentration of target diolefin and an increased concentration of secondary diolefin and CPD.

8. The method of claim 7 wherein the prefractionation conditions comprise:
a prefractionation temperature of from about 40° C. to about 68° C.; and
a prefractionation pressure of from about 35 kPa to about 345 kPa.

9. The method of claim 7 further comprising subjecting the crude secondary diolefin feedstock to crude secondary diolefin/CPD dimerization conditions effective to produce a raw secondary diolefin stream comprising an increased concentration of DCPD.

10. The method of claim 9 wherein the crude secondary diolefin/CPD dimerization conditions comprise:
a crude secondary diolefin/CPD dimerization temperature of from about 68° C. (155° F.) to about 82° C. (180° F.);
a crude secondary diolefin/CPD dimerization pressure of from about 103 kPa (15 psig) to about 345 kPa (50 psig); and,
a crude secondary diolefin/CPD dimerization residence time of from about 5 hours to about 10.5 hours.

11. The method of claim 10 further comprising subjecting the raw secondary diolefin stream to post-fractionation conditions effective to separate a resins grade secondary diolefin product and a low purity DCPD product.

12. The method of claim 11 wherein the post-fractionation conditions comprise:
a post-fractionation temperature of from about 55° C. to about 68° C.; and
a post-fractionation pressure of from about 55 kPa to about 345 kPa.

13. The method of any of claims 1-12 wherein the target diolefin is isoprene and the secondary diolefin is piperylenes.

14. A method of treating a hydrocarbon stream comprising cyclopentadiene (CPD) and isoprene, the method comprising:
providing a crude C5 feedstock comprising an initial CPD concentration, an initial isoprene concentration, and an initial piperylenes (PIPS) concentration;
subjecting the crude C5 feedstock to preseparation conditions effective to separate (a) a crude isoprene feedstock having a decreased concentration of CPD and an increased isoprene concentration, and (b) a CPD dimerization feedstock having a decreased isoprene concentration and an increased CPD concentration that is 1.5 or more times the initial CPD concentration;
subjecting the CPD dimerization feedstock to CPD dimerization conditions effective to produce a raw DCPD stream comprising 20 wt. % or more DCPD; and,
separating a high purity DCPD product stream comprising 90 wt. % or more DCPD from the raw DCPD stream.

15. The method of claim 14 wherein:
the preseparation conditions comprise a preseparation temperature of from about 40° C. to about 50° C.;
a preseparation pressure of from about 34.5 kPa to about 110 kPa.

16. The method of claim 14 wherein the decreased isoprene concentration is 2.9 or more times less than the initial isoprene concentration.

17. The method of claim 14 wherein the ratio of the concentration of CPD to the concentration of isoprene in the CPD dimerization feedstock is about 7 or more.

18. The method of claim 14 wherein the CPD dimerization conditions comprise:
a CPD dimerization temperature of from about 40.5° C. to about 71° C.;
a CPD dimerization pressure of from about 103 kPa to about 345 kPa; and,
a CPD dimerization residence time of from about 5 hours to about 10.5 hours.

19. The method of claim 18 wherein the DCPD separation conditions comprise:
a DCPD separation temperature of from about 50° C. to about 170° C.; and
a DCPD separation pressure of from about 28 kPa to about 55 kPa.

20. The method of claim 14 further comprising subjecting the crude isoprene feedstock to prefractionation conditions effective to separate (a) a raw isoprene stream comprising a further increased concentration of isoprene and a further decreased concentration of CPD and PIPS, and (b) a crude PIPS feedstock comprising a decreased concentration of isoprene and an increased concentration of PIPS and CPD.

21. The method of claim 20 wherein the prefractionation conditions comprise:
a prefractionation temperature of from about 40° C. to about 68° C.; and
a prefractionation pressure of from about 35 kPa to about 345 kPa.

22. The method of claim 21 further comprising subjecting the crude PIPS feedstock to crude PIPS/CPD dimerization conditions effective to produce a raw PIPS stream comprising an increased concentration of DCPD.

23. The method of claim 22 wherein the crude PIPS/CPD dimerization conditions comprise:
a crude PIPS/CPD dimerization temperature of from about 68° C. to about 82° C.;
a crude PIPS/CPD dimerization pressure of from about 103 kPa to about 345 kPa; and,
a crude PIPS/CPD dimerization residence time of from about 5 hours to about 10.5 hours.

24. The method of claim 22 further comprising subjecting the raw PIPS stream to post-fractionation conditions effective to separate a resins grade PIPS product and a low purity DCPD product.

25. The method of claim 24 wherein the post-fractionation conditions comprise:
a post-fractionation temperature of from about 55° C. to about 68° C.; and
a post-fractionation pressure of from about 55 kPa to about 345 kPa.
